(19) United States
(12) Patent Application Publication
(10) Pub. No.: US 2009/0036525 A1
(43) Pub. Date: Feb. 5, 2009

(54) Non-Natural Amino Acid Derivatives

(75) Inventor: Christopher Hobbs, London (GB)

Correspondence Address: BANNER & WITCOFF, LTD., 1100 13th STREET, N.W., SUITE 1200, WASHINGTON, DC, 20005-4051, US

(73) Assignee: POXIMAGEN LTD., London (GB)

(21) Appl. No.: 12/278,422

(22) PCT Filed: Jan. 23, 2007
(86) PCT No.: PCT/GB2007/000205

§ 371 Date: Aug. 6, 2008

(30) Foreign Application Priority Data
Feb. 11, 2006 (GB) 0602799.9

(51) Int. Cl. A61K 31/235; C07C 229/00; A61K 31/195; C07C 59/00

(52) U.S. Cl. 514/533; 514/542; 560/42; 514/563; 562/471

(57) ABSTRACT

Compounds of formula (I) have activity in alleviating the effects of impaired dopaminergic signaling, for example in the treatment of Parkinsons Disease:

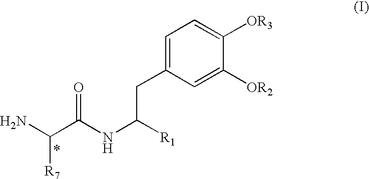

wherein:

R₁ is a carboxyl, carboxyl ester, or carboxamide group; R₂ and R₃ are independently hydrogen, or a group —C(═O)R₆ or —C(═O)OR₆ wherein R₆ is C₁-C₆ alkyl, or a group —CH₂Q wherein Q is an optionally substituted monocyclic cycloalkyl or heterocyclyl ring of 3 to 6 ring atoms; R₇ is (i) optionally substituted phenyl or monocyclic heteroaryl, or (ii) a radical of formula —CHR₄R₅; R₄ is (a) optionally substituted C₁-C₄ alkyl, C₁-C₄ alkoxy, C₂-C₄ alkenyl, C₂-C₄ alkenyloxy, or C₂-C₄ alkynyl, or (b) —CH₂XCH₃, —CH₂CH₂XCH₃, or —CH₂XCH₂CH₃, wherein X is —O—, S, or —NR₇ wherein R₇ is hydrogen, methyl or ethyl; or —CH₂Q or CH₂OQ wherein Q is as defined in relation to R₆; and R₅ is hydrogen, methyl, ethyl, or methyl substituted by 1, 2 or 3 fluoro atoms; or R₄ and R₅ taken together with the carbon atom to which they are attached form an optionally substituted carbocyclic or heterocyclic ring of 3 to 6 ring atoms, optionally fused to a second, optionally substituted, carbocyclic or heterocyclic ring or 3 to 8 ring atoms; PROVIDED THAT the group R₇ is not the side chain of a natural amino acid.

The present invention relates to compounds which diminish the symptoms of dopamine deficiency.

Dopamine is a substance produced naturally by neurons in the basal ganglia of the brain that allows smooth, coordinated control of voluntary movement. Loss of, or impairment of, dopamine-producing neurons in the brain is implicated in Parkinson's disease and related Parkinson-plus syndromes. These conditions respond to dopamine replacement therapy. Other conditions, for example, Restless Legs Syndrome (RLS) also respond to dopamine replacement therapy.

Parkinson's disease is a progressive neurodegenerative disorder that affects neuronal cells in the substantia nigra in the mid-brain. It is an age-related disorder of the central nervous system primarily attacking people over the age of 60. Approximately one out of every 500 people contract the illness and approximately one out of every 100 people over the age of 60 contract the illness. As indicated above, Parkinson's disease is thought to be caused by a deficiency of dopamine. The common symptoms include tremor, stiffness (or rigidity) of muscles, slowness of movement (bradykinesia) and loss of balance (postural dysfunction). Parkinson's disease is one of the most prevalent neurodegenerative illnesses. The natural history of the disease is progressive and from 10-15 years from onset of the disease becomes disabling in most patients.

Parkinson's disease is largely sporadic and referred to as idiopathic in nature. Forms of the illness due to vascular incidents and to toxin exposure also exist. Rare familial forms of the illness also exist.

Many treatments have been tried since James Parkinson first described the condition in 1817. Current therapy for Parkinson's Disease is based on dopamine replacement therapy based on the use of the dopamine precursor levodopa (or L-dopa) or dopaminergic compounds. L-dopa is highly effective in reversing the motor symptoms of the illness but on chronic treatment and with disease progression, its effectiveness declines. The duration of drug response is reduced and unpredictable fluctuations in movement occur. Treatment is associated with therapy limiting side effects which include involuntary movements (dyskinesia) and psychosis.

RLS is a neurosensorimotor disorder with parestethesias, sleep disturbances and, in most cases, periodic limb movements of sleep (PLMS). Two forms of RLS appear to exist: the idiopathic and the uremic form. RLS is characterised by (1) a desire to move the legs, usually associated with paresthesias/dysesthesias, (2) motor restlessness, (3) worsening or exclusive presence of symptoms at rest (i.e. lying, sitting) with at least partial or temporary relief by activity, and (4) worsening of symptoms during the evening or night.

The present invention provides compounds which are active as dopaminergic compounds or as compounds which or as compounds which diminish the symptoms of dopamine deficiency.

According to the invention, there is provided a compound of formula (I) or a salt, hydrate or solvate thereof:

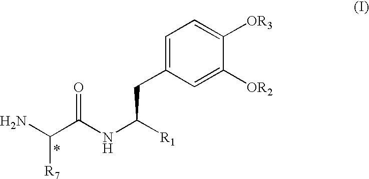

wherein:

R₁ is a carboxyl, carboxyl ester, or carboxamide group;

R₂ and R₃ are independently hydrogen, or a group —C(═O)R₆ or —C(═O)OR₆ wherein R₆ is C₁-C₆ alkyl, or a group —CH₂Q wherein Q is an optionally substituted monocyclic cycloalkyl or heterocyclyl ring of 3 to 6 ring atoms;

R₇ is (i) optionally substituted phenyl or monocyclic heteroaryl, or (ii) a radical of formula —CHR₄R₅;

R₄ is
(a) optionally substituted C₁-C₄ alkyl, C₁-C₄ alkoxy, C₂-C₄ alkenyl, C₂-C₄ alkenyloxy, or C₂-C₄ alkynyl, or
(b) —CH₂XCH₃, —CH₂CH₂XCH₃, or —CH₂XCH₂CH₃, wherein X is —O—, S, or —NR₇ wherein R₇ is hydrogen, methyl or ethyl; or —CH₂Q or CH₂OQ wherein Q is as defined in relation to R₆; and

R₅ is hydrogen, methyl, ethyl, or methyl substituted by 1, 2 or 3 fluoro atoms; or

R₄ and R₅ taken together with the carbon atom to which they are attached form an optionally substituted carbocyclic or heterocyclic ring of 3 to 6 ring atoms, optionally fused to a second, optionally substituted, carbocyclic or heterocyclic ring or 3 to 8 ring atoms;

PROVIDED THAT the group R₇ is not the side chain of a natural amino acid.

The compounds of the invention may be regarded as amino acid derivatives of L-dopa (2-amino-3-(3,4-dihydroxyphenyl)-propanoic acid) or L-dopa-like compounds, wherein the former (to the left of the wavy line in formula (IA)) is linked to the latter (to the right of the wavy line in formula (IA)) by a peptide bond (intersected by the wavy

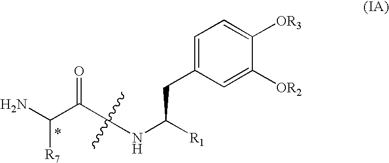

line in formula (IA)):

The amino acid which acylates the amino group of the L-dopa part is characterised by di-substitution on its alpha carbon atom.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “(Cₐ-C_{b})alkenyl” means a straight or branched chain alkenyl moiety having from a to b carbon atoms having at least one double bond of either E or Z stereochemistry where applicable. Thus when a is 2 and b is 6, the term includes, for example, vinyl, allyl, 1- and 2-butenyl and 2-methyl-2-propenyl.

As used herein the term “C₂-C₆ alkynyl” refers to straight chain or branched chain hydrocarbon groups having from a to b carbon atoms and having in addition one triple bond. Thus when a is 2 and b is 6, the term includes, for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the unqualified term “carbocyclic” refers to a mono-, bi- or tricyclic radical having up to 16 ring atoms, all of which are carbon, and includes aryl and cycloalkyl.

As used herein the unqualified term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzothienyl, furyl, benzofuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in its non-aromatic meaning relates to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzofuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with up to four compatible substituents, each of which independently may be, for example, (C₁-C₆)alkyl, (C₃-C₆) cycloalkyl, (C₁-C₆)alkoxy, hydroxy, hydroxy(C₁-C₆)alkyl, mercapto, mercapto(C₁-C₆)alkyl, (C₁-C₆)alkylthio, phenyl, monocyclic heterocyclic, benzyl, phenoxy, halo (including fluoro, bromo and chloro), trifluoromethyl, trifluoromethoxy, nitro, nitrile —CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂—, —NHR^{A}, —NR^{A}R^{B}, —OCONH₂, OCONHR^{A} —OCONR^{A}R^{B}, —NHCOR^{A}, —NHCOOR^{A}, —NR^{B}COOR^{A}, —NHS₂OR^{A}, —NRBSO₂OH, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆) cycloalkyl, phenyl, benzyl or monocyclic heterocyclic having 5 or 6 ring atoms, or R^{A} and R^{B} when attached to the same nitrogen may, together with that nitrogen, form a 4- to 6-membered ring containing that nitrogen. An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesulfonic, glutamic, lactic, and mandelic acids and the like.

In the compounds of the invention, carbon atom to which R₁ is attached is assymetric, and the stereochemistry at that centre is as shown in formula (I). However, the compounds of the invention may contain one or more additional chiral centres, because of the presence of asymmetric carbon atoms, and they can exist as a number of diastereoisomers with R or S stereochemistry at each chiral centre. The invention includes all such diastereoisomers and mixtures thereof.

The Group R₁

R₁ may be a carboxyl group (—COOH), a carboxyl ester group or a carboxamide group. Compounds wherein R₁ is a carboxyl group form one presently preferred subclass.

Examples of carboxyl ester groups R₁ include those of formula —COOR^{C} wherein R^{C} is a C₁-C₆ alkyl or C₂-C₆ alkenyl group. A presently preferred carboxyl ester group is the methyl ester —COOCH₃.

Examples of carboxamide groups R₁ include those of formula —CONR^{B}(Alk)ₙR^{A} wherein
Alk is an optionally substituted divalent C₁-C₆ alkylene, or C₂-C₆ alkenylene or C₂-C₆ alkynylene radical,
n is 0 or 1,
R^{B} is hydrogen or a C₁-C₆ alkyl or C₂-C₆ alkenyl group,
R^{A} is hydrogen, hydroxy or optionally substituted carbocyclic or heterocyclyl,
or R^{A} and R^{B} taken together with the nitrogen to which they are attached form an N-heterocyclic ring which may optionally contain one or more additional hetero atoms selected from O, S and N, and which may optionally be substituted on one or more ring C or N atoms.

Thus, in carboxamide groups R₁ of formula —CONR^{B}(Alk)ₙR^{A}, Alk may be optionally substituted —CH₂—, —CH₂CH₂—, —CH₂CH₂CH₂—, —CH₂CH═CH—, or —CH₂CCCH₂—; R^{B} may be hydrogen or methyl, ethyl, n- or iso-propyl, or allyl; R^{A} may be hydroxy or optionally substituted phenyl, 3,4-methylenedioxyphenyl, pyridyl, furyl, thienyl, N-piperazinyl, or N-morpholinyl; or R^{A} and R^{B} taken together with the nitrogen to which they are attached form an N-heterocyclic ring which may optionally contain one or more additional hetero atoms selected from O, S and N, and which may optionally be substituted on one or more ring C or N atoms.

A presently preferred carboxamide group R₁ is —CONH₂.

The Groups R₂ and R₃

In the compounds of the invention R₂ and R₃ may be the same or different

In a presently preferred subclass of compounds of the invention R₂ and R₃ are each hydrogen.

In another subclass, R₂ and R₃ are independently —C(═O)R₆ or —C(═O)OR₆ wherein R₆ is methyl, ethyl, n- or isopropyl, tert-butylmethyl, or benzyl which is optionally substituted in the phenyl ring thereof.

The Group R₇

When R₇ is a radical of formula —CHR₄R₅ a particular subclass of compounds of the invention has R₄ as optionally substituted ethyl, n- or iso-propyl, n-, iso- or tert butyl, phenyl, naphthyl, benzyl, cyclopropyl, cyclobutyl, cyclopentyl, cyclohexyl, cyclopropylmethyl, cyclobutylmethyl, cyclopentylmethyl, cyclohexylmethyl, pyridyl, pyridylmethyl, piperidinyl, piperazinyl or morpholinyl. In such cases, and indeed in any case when R₇ is a radical of formula —CHR₄R₅, R₅ may be hydrogen.

When R₇ is a radical of formula —CHR₄R₅, another subclass of compounds of the invention has R₄ and R₅ taken together with the carbon atom to which they are attached forming an optionally substituted cyclopropyl, cyclobutyl, cyclopentyl, cyclohexyl, or piperidinyl ring.

In yet another subclass of compounds of the invention R₇ is an optionally substituted phenyl, pyridyl, thienyl, furyl, or pyrrolyl ring.

Optional Substituents

When any of the variable substituents in compounds (I) of the invention is optionally substituted, such optional substituents may be selected from, for example, methyl, trifluoromethyl, methoxy, trifluoromethoxy, cyclopropyl, halogen, cyano, hydroxy, mercapto, oxo, —NH₂, —NHR^{A}, or —NR^{A}R^{B} wherein R^{A} and R^{B} are independently methyl or ethyl.

Stereochemistry

In the compounds of the invention, it is currently preferred that the stereochemical orientation of the bond marked * is S.

Examples of specific compounds of the invention include those of the examples herein.

Synthetic Routes

There are multiple synthetic strategies for the synthesis of the compounds (I) with which the present invention is concerned, but all rely on chemistry known to the synthetic organic. Compounds according to formula (I) can be synthesised according to procedures described in the standard literature and are well-known to the one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “SciFinder” or “Beilstein”.

In general the compounds of the invention are accessible by well known methods of peptide synthesis whereby an acylating derivative of an amino acid (II) is reacted with the amino group of an amino acid of formula (III)

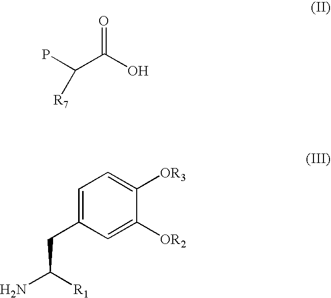

wherein P is a protected amino group and R₁-R₇ are as defined in relation to formula (I) above, to form the desired peptide bond between the two reactants, and the protected amino group of reactant (II) is deprotected.

Pharmaceutical Utility

As mentioned above, many of the compounds of the invention are cleaved in the body to release L-dopa or L-dopa precursors. L-dopa itself is metabolised in the gut, the gut membrane, plasma, kidney and the liver and this significantly reduces its bioavailability and increases intersubject variability in the resulting blood levels of L-dopa. The present compounds have a different pharmacokinetic profile from L-dopa itself, due to the time over which cleavage occurs and L-dopa is released.

Hence the compounds of the present invention are useful in a method of treatment of a condition associated with impaired dopaminergic signalling in a subject, comprising administering to the subject an amount of the compound effective to reduce such impairment. The compounds are also useful in the preparation of a composition for treatment of a condition associated with impaired dopaminergic signalling. Examples of such conditions include Parkinson's disease, or Restless Legs Syndrome, as well as Tourette's syndrome, attention deficit hyperactive disorder, generation of pituitary tumours, a parkinson-plus syndrome, levodopa responsive dystonia, dyskinesia, periodic movements in sleep, dysphagia or neuroleptic malignant syndrome.

Typical examples of Parkinson's disease which can be treated with the compounds of the invention include sporadic Parkinson's disease, familial forms of Parkinson's disease and post-encephalitic Parkinsonism.

Typical examples of Parkinson-plus syndromes which can be treated with the compounds of the invention include progressive supranuclear palsy and multiple system atrophy.

Typically, the dyskinesia is L-dopa-induced dyskinesia.

The compounds of the invention may be administered in a variety of dosage forms. Thus, they can be administered orally, for example as tablets, capsules, troches, lozenges, aqueous or oily suspensions, dispersible powders or granules. The compounds can be administered in a sublingual formulation, for example a buccal formulation. The compounds of the invention may also be administered parenterally, whether subcutaneously, intravenously, intramuscularly, intrasternally, transdermally, by inhalation (e.g. intranasally) or by infusion techniques. The compounds may also be administered as suppositories. Typically, the compounds of the invention are administered orally or by inhalation (e.g. intranasally). Preferably, the compounds of the invention are administered orally. More preferably, the compounds of the invention are administered as a tablet or capsule.

The present invention further provides a pharmaceutical composition containing a compound of formula (I), or a pharmaceutically acceptable salt thereof, as defined above, and a pharmaceutically acceptable carrier.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; desegregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol. Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Since the compounds of the invention are preferably administered orally, the present invention further provides a pharmaceutical composition containing a compound of formula (I), or a pharmaceutically acceptable salt thereof, as defined above, and a pharmaceutically acceptable carrier in the form of a capsule or tablet.

Solutions for injection or infusion may contain as carrier, for example, sterile water or preferably they may be in the form of sterile, aqueous, isotonic saline solutions.

The compounds of the present invention may also be administered with a peripheral decarboxylase inhibitor. The present invention therefore provides a pharmaceutical composition containing a compound of formula (I), or a pharmaceutically acceptable salt thereof, as defined above, a peripheral decarboxylase inhibitor and a pharmaceutically acceptable carrier or diluent. Typically the peripheral decarboxylase inhibitor is carbidopa or benserazide. Preferably the peripheral decarboxylase inhibitor is carbidopa.

Also provided is a product comprising (a) a compound of the formula (I), or a pharmaceutically acceptable salt thereof, as defined above and (b) a peripheral decarboxylase inhibitor as defined above, for simultaneous separate or sequential use in the treatment of the human or animal body.

Further, said medicament is typically for co-administration with a peripheral decarboxylase inhibitor defined above.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial. However, it is expected that a typical dose will be in the range from about 0.001 to 50 mg per kg of body weight.

The following examples illustrate the invention:

LC-MS Method

The system used to obtain LC-MS data comprised a Waters Alliance 2695 quaternary HPLC, Waters 996 Photo Diode Array (PDA) detector and Waters ZQ 2000 single quadrupole mass spectrometer. The ZQ can acquire data simultaneously in positive and negative electrospray ionisation modes. ZQ Mass Spectrometer

| | | | |
|---|---|---|---|
| Capillary | 3.3 kV/−3.0 kV | Cone | 40 V/−40 V |
| Extractor | 5 V/−5 V | Source Temp | 110° C. |
| Desolvation Temp | 400° C. | Cone Gas | 40 L/Hr |
| Desolvation Gas | 350 L/Hr | Multiplier | 500 V/−500 V |

Data were acquired in a full scan from 80 to 1000 m/z

| | |
|---|---|
| Scan duration | 0.80 s |
| InterScan delay | 0.20 s |

HPLC

The reverse phase separation was carried out on a Zorbax XDB C8 150×4.6 mm with 5 μm silica from Agilent for both the low and neutral pH methods.

| | |
|---|---|
| Injection Volume | 10 μL |
| UV data | 220 to 400 nm |
| Sample Temperature | 20° C. |
| Column Temperature | 30° C. |
| Flow Rate | 1.0 mL/min |
| Split to ZQ | 0.3 mL/min |

LC Method (approximately pH 3.2)

| | |
|---|---|
| Solvent A | Water/10 mM NH₄HCO₂/0.1% formic acid |
| Solvent B | 95% CH₃CN/5% A/0.1% formic acid |

Gradient Program for LC Method

| Time | A % | B % | C % | D % | Flow | Curve |
|---|---|---|---|---|---|---|
| 0.00 | 95.0 | 5.0 | 0.0 | 0.0 | 1.000 | 1 |
| 1.00 | 95.0 | 5.0 | 0.0 | 0.0 | 1.000 | 6 |
| 11.00 | 5.0 | 95.0 | 0.0 | 0.0 | 1.000 | 6 |
| 14.20 | 5.0 | 95.0 | 0.0 | 0.0 | 1.000 | 6 |
| 14.50 | 95.0 | 5.0 | 0.0 | 0.0 | 1.000 | 6 |
| 15.00 | 95.0 | 5.0 | 0.0 | 0.0 | 1.000 | 6 |

### EXAMPLE 1

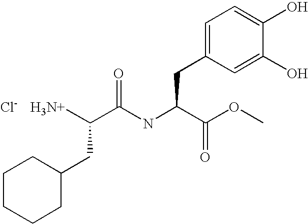

### (S)-2-Cyclohexyl-1-[(S)-2-(3,4-dihydroxyphenyl)-1-methoxycarbonylethyl-carbamoyl]-ethylammonium chloride

Step 1

To a solution of (S)-2-(3,4-dihydroxyphenyl)-1-methoxycarbonylethylammonium chloride (0.50 g, 2 mmol) in dichloromethane (10 mL) was added triethylamine (0.224 g, 2.22 mmol) DMF (2 mL), 2-tert-butoxycarbonylamino-2-methylpropionic acid (0.532, 2.42 mmol), and HOBt (0.30 g, 2.22 mmol). The mixture was stirred for 20 min. EDC (0.426 g, 2.22 mmol) was added and stirring was continued for 16 h. AcOH (0.3 mL) was added. The mixture was washed with saturated NaHCO₃ solution and saturated NaCl solution. The organic layer was dried over Na₂SO₄. The sodium Na₂SO₄ was removed by filtration and the filtrate evaporated in vacuo. The residue was purified by column chromatography, eluting with ethyl acetate/hexane 3:2 to afford a colorless oil (0.82 g).

Step 2

The product of Step 1 (0.40 g) was suspended in dichloromethane (5 mL) and the suspension cooled in an ice-water bath. A solution of HCl in dioxane (4M, 2.0 mL) was added and the mixture stirred at 20° C. for 5 h. Ether was added and the mixture stirred for a further 1 h. The resulting precipitate was collected by filtration and dried in vacuo to afford a colourless solid (0.135 g). HPLC retention time 6.12 min. Mass spectrum (ES+) m/z 365 (M+H).

### EXAMPLE 2

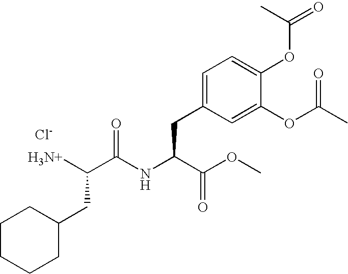

### (S)-2-Cyclohexyl-1-[(S)-2-(3,4-diacetoxyphenyl)-1-methoxycarbonyl-ethylcarbamoyl]-ethylammonium chloride

The product of Example 1 Step 1 (0.010 g) was treated with acetic acid (3 mL) and heated to 40° C. HCl (g) was bubbled through the solution. Acetyl chloride (2 mL) was added dropwise. The mixture was cooled to 20° C. and stirred for 16 h. Ether was added and the precipitate was collected by filtration to afford a colourless solid (0.080 g). HPLC retention time 7.25 min. Mass spectrum (ES+) m/z 449 (M+H).

### EXAMPLE 3

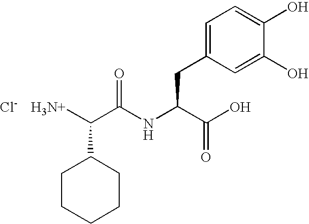

### (S)—C—[(S)-1-Carboxy-2-(3,4-dihydroxyphenyl)-ethylcarbamoyl]-C-cyclohexyl-methylammonium chloride

Step 1

3,4-Dihydroxy-L-phenylalanine (15.0 g, 0.076 mole) was suspended in benzyl alcohol (381 mL). This suspension was then cooled with an ice bath to 5° C. and treated with thionyl chloride (76.2 mL). The resulting solution was heated to between 95° C. and 100° C. for 5 h, in a nitrogen atmosphere. The suspension was cooled to 20° C. and diluted with 1.5 L of dry ether to yield a solid precipitate. The suspension was stirred at 20° C. temperature for 16 h, filtered, and then washed with ether and dried in a vacuum oven to afford a colourless solid (7.7 g).

Step 2

A glacial acetic acid (66 ml) suspension containing 3,4-dihydroxy-L-phenylalanine-benzyl ester hydrochloride (3.23 g, 0.01 mol) was warmed to 110° C. and HCl(g) was bubbled through the mixture for a 4 min. The solution was cooled to 45° C. and acetyl chloride (10.1 ml) was added. This solution was stirred at 20° C. temperature for 16 h and then, diluted with dry ether (150 mL). The precipitate was filtered and dried under vacuum to afford colourless crystals (3.4 g).

Step 3

To a solution of 1(S)-benzyloxycarbonylaminocyclohexyl-acetic acid (1 mmol) in dichloromethane was added the product of Step 2 (1 mmol) at 0° C. followed by PyBOP (1 mmol) and DIPEA (2 mmol). The reaction was allowed to warm to 20° C. and stirred for 12 h. The reaction mixture was washed with 1 M HCl followed by satd. aq. NaHCO₃. The organic layer was dried and concentrated in vacuo to obtain the crude product, which was purified by flash chromatography eluting with ethyl acetate:hexane (stepwise gradient from 100% hexane to 100% ethyl acetate).

Step 4

The product of Step 3 (1 mmol) was dissolved in THF (20 mL) and 1M HCl (5 mL) was added. This was followed by addition of 10% Pd/C (50% wet, 600 mg). The resulting suspension was hydrogenated at 1 atm H₂ for 24 h. The catalyst was removed by filtration and the filtrate was concentrated in vacuo. The residue was purified by flash chromatography eluting with 10% MeOH/dichloromethane. The product was dissolved in MeCN:H₂O (1:1) and lyophilized to afford a yellow solid (0.104 g). HPLC retention time 4.46 min. Mass spectrum (ES+) m/z 337 (M+H).

### EXAMPLE 4

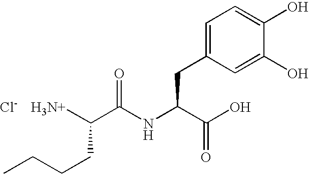

### (S)-1-[(S)-1-Carboxy-2-(3,4-dihydroxyphenyl)-ethylcarbamoyl]-pentyl-ammonium chloride

An analogous procedure to Example 3 was followed starting from (S)-2-benzyloxy-carbonylaminohexanoic acid (1 mmol) which afforded a grey/green solid (0.133 g). HPLC retention time 4.03 min. Mass spectrum (ES+) m/z 311 (M+H).

### EXAMPLE 5

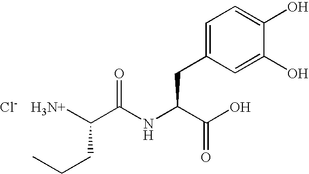

### (S)-1-[(S)-1-Carboxy-2-(3,4-dihydroxyphenyl)-ethylcarbamoyl]-butyl-ammonium chloride

An analogous procedure to Example 3 was followed starting from (S)-2-benzyloxycarbonylaminopentanoic acid (1 mmol) which afforded a dark green oil (0.11 g). HPLC retention time 2.61 min. Mass spectrum (ES+) m/z 297 (M+H).

### EXAMPLE 6

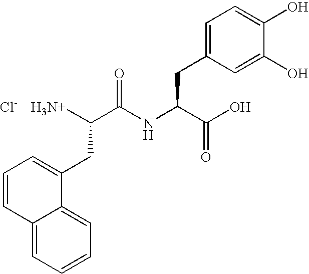

### (S)-1-[(S)-1-Carboxy-2-(3,4-dihydroxyphenyl)-ethylcarbamoyl]-2-naphthalen-1-ylethylammonium chloride

Step 1

To a solution of (S)-2-tert-butoxycarbonylamino-3-naphthalen-1-yl-propionic acid (1 mmol) in dichloromethane at 0° C. was added the product of Example 3 Step 1 (1 mmol) followed by PyBOP (1 mmol) and DIPEA (2 mmol). The reaction was allowed to warm to 20° C. and stirred at 20° C. for 12 h. The reaction mixture was washed with 1 M HCl followed by satd. aq. NaHCO₃. The organic layer was dried and concentrated to obtain the crude product, which was purified by flash chromatography eluting with ethyl acetate:hexane (stepwise gradient from 100% hexane to 100% ethyl acetate).

Step 2

The product of Step 1 (1 mmol) was dissolved in THF (20 mL) and 10% Pd/C (50% wet, 600 mg) was added. The resulting suspension was hydrogenated at 1 atm H₂ for 24 h. The catalyst was removed by filtration and the filtrate was concentrated in vacuo. The product was purified by flash chromatography eluting with 5% MeOH/dichloromethane. The product was dissolved in dioxane (10 mL), cooled to 15° C. and saturated with HCl (g). The reaction mixture was stirred for 12 h after which the solvent was removed in vacuo. The residue was purified by flash chromatography eluting with 10% MeOH/dichloromethane to afford a brown solid (0.110 g). HPLC retention time 5.43 min. Mass spectrum (ES+) m/z 395 (M+H).

### EXAMPLE 7

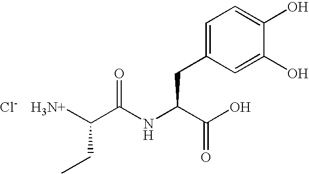

### (S)-1-[(S)-1-Carboxy-2-(3,4-dihydroxyphenyl)-ethylcarbamoyl]-propyl-ammonium chloride

An analogous procedure to Example 6 was followed starting from (S)-2-benzyloxy-carbonylaminobutyric acid (1 mmol) which afforded a beige solid (0.105 g). HPLC retention time 1.59 min. Mass spectrum (ES+) m/z 283 (M+H).

### EXAMPLE 8

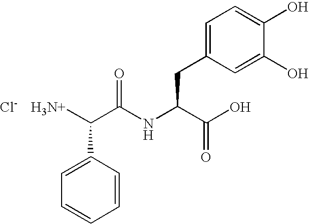

### (S)—C—[(S)-1-Carboxy-2-(3,4-dihydroxyphenyl)-ethylcarbamoyl]-C-phenylmethyl-ammonium chloride

An analogous procedure to Example 3 was followed starting from (S)-benzyloxy-carbonylaminophenylacetic acid (1 mmol) which afforded a pale yellow solid (0.100 g). HPLC retention time 4.27 min. Mass spectrum (ES+) m/z 331 (M+H).

### EXAMPLE 9

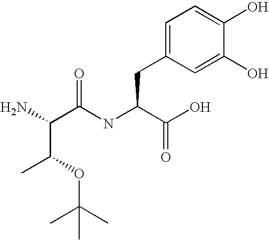

### (S)-2-((2S,3R)-2-Amino-3-tert-butoxybutyrylamino)-3-(3,4-dihydroxyphenyl)-propionic acid

Step 1

To a solution of (2S,3R)-3-tert-butoxy-2-tert-butoxycarbonylaminobutyric acid (1 mmol) in dichloromethane was added the product of Example 3 Step 1 (1 mmol) at 0° C. followed by PyBOP (1 mmol) and DIPEA (2 mmol). The reaction was allowed to warm to 20° C. and stirred at 20° C. for 12 h. The reaction mixture was washed with 1 M HCl followed by satd. aq. NaHCO₃. The organic layer was dried and concentrated in vacuo. The residue was purified by flash chromatography eluting with ethyl acetate:hexane (stepwise gradient from 100% hexane to 100% ethyl acetate).

Step 2

The product of Step 1 (1 mmol) was dissolved in THF (20 mL) and 10% Pd/C (50% wet, 600 mg) was added. The resulting suspension was hydrogenated at 1 atm H₂ for 24 h. The catalyst was removed by filtration and the filtrate was concentrated in vacuo. The residue was purified by flash chromatography eluting with 10% MeOH/dichloromethane. The product was dissolved in MeCN:H₂O (1:1) and lyophilized to afford a light brown solid (0.100 g). HPLC retention time 4.68 min. Mass spectrum (ES−) m/z 353 (M−H).

### EXAMPLE 10

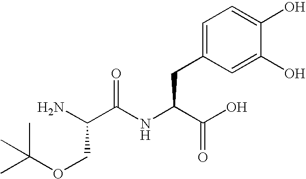

### (S)-2-((S)-2-Amino-3-tert-butoxypropionylamino)-3-(3,4-dihydroxyphenyl)-propionic acid

An analogous procedure to Example 9 was followed starting (S)-2-benzyloxy-carbonylamino-3-tert-butoxypropionic acid (1 mmol) which afforded a brown solid (0.155 g). HPLC retention time 4.3 min. Mass spectrum (ES+) m/z 285 (M+H).

### EXAMPLE 11

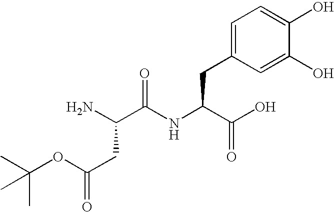

### (S)-3-Amino-N—[(S)-1-carboxy-2-(3,4-dihydroxyphenyl)-ethyl]-succinamic acid tert-butyl ester

An analogous procedure to Example 9 was followed starting (S)-2-benzyloxycarbonylamino-N-tert-butylsuccinamic acid (1 mmol) which afforded a beige solid (0.098 g). HPLC retention time 4.79 min. Mass spectrum (ES+) m/z 312 (M−tBu+H).

### EXAMPLE 12

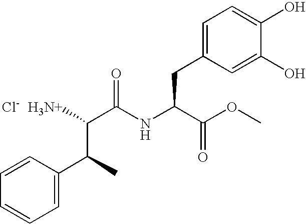

### (1S,2S)-1-[(S)-2-(3,4-Dihydroxyphenyl)-1-methoxycarbonylethylcarbamoyl]-2-phenylpropylammonium chloride

Step 1

A mixture of (2S,3S)-2-tert-butoxycarbonylamino-3-phenylbutyric acid (0.50 g, 1.79 mmol) and (S)-2-(3,4-dihydroxyphenyl)-1-methoxycarbonylethylammonium chloride (0.443 g, 1.79 mmol) in dichloromethane (35 mL) was stirred at 20° C. Triethylamine (0.50 mL, 3.58 mmol) was added, the reaction mixture was stirred for 1 min and HBTU (0.679 g, 1.79 mmol) was added. Stirring was continued at 20° C. for 16 h. Dimethylacetamide was added and stirring was continued for a further 16 h. The reaction mixture was diluted with dichloromethane, washed with water, dried and evaporated in vacuo to give a yellow oil which was purified by flash column chromatography eluting with ethyl acetate/hexane (1:2) to afford a colourless solid (0.23 g).

Step 2

An analogous procedure to Example 1 Step 2 was followed starting from the product of Step 1 (0.23 g, 0.487 mmol) which afforded an off-white solid (0.23 g). HPLC retention time 5.77 min. Mass spectrum (ES+) m/z 373 (M+H).

### EXAMPLE 13

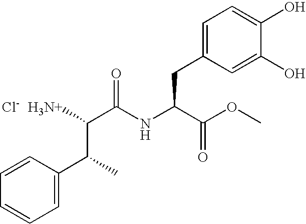

### (1S,2R)-1-[(S)-2-(3,4-Dihydroxyphenyl)-1-methoxycarbonyl-ethylcarbamoyl]-2-phenylpropylammonium; chloride

Step 1

A mixture of (2S,3R)-2-tert-butoxycarbonylamino-3-phenylbutyric acid (0.50 g, 1.79 mmol) and (S)-2-(3,4-dihydroxyphenyl)-1-methoxycarbonylethylammonium chloride (0.443 g, 1.79 mmol) in dichloromethane (35 mL) was stirred at 20° C. Triethylamine (0.50 mL, 3.58 mmol) was added, the reaction mixture was stirred for 1 min and HBTU (0.679 g, 1.79 mmol) was added. Stirring was continued at 20° C. for 16 h. Dimethylacetamide was added and stirring was continued for a further 16 h. The reaction mixture was diluted with dichloromethane, washed with water, dried and evaporated in vacuo to give a yellow oil which was purified by flash column chromatography eluting with ethyl acetate/hexane (1:2) to afford a colourless oil (0.26 g).

Step 2

An analogous procedure to Example 1 Step 2 was followed starting from the product of Step 1 (0.26 g, 0.55 mmol) which afforded a pale yellow solid (0.0.105 g). HPLC retention time 5.68 min. Mass spectrum (ES+) m/z 373 (M+H).

TABLE
Summary of LCMS data

| Example | retention | (ES+) m/z |
|---|---|---|
| no. | time | (M + H) |
| 1 | 6.12 | 365 |
| 2 | 7.25 | 449 |
| 3 | 4.46 | 337 |
| 4 | 4.03 | 311 |
| 5 | 2.61 | 297 |
| 6 | 5.43 | 395 |
| 7 | 1.59 | 283 |
| 8 | 4.27 | 331 |
| 9 | 4.68 | 299 (M- |
| 10 | 4.3 | 285 |
| 11 | 4.79 | 312 (M- |
| 12 | 5.77 | 373 |
| 13 | 5.68 | 373 |

Biological Results

Compounds of the examples above were tested in the following animal model of dopamine deficiency behaviour:

Assessment of Activity in 6-OHDA-Lesioned Rats
Animals Male Wistar rats, 250 g, Harlan Ltd.
Housing Animals were housed in groups of 4 on a 12-h light-dark cycle with an environment of 50% humidity and temperature of 21±2° C. in accordance with Animals (Scientific Procedures) Act 1996 Home Office regulations. Rats had access to food and water ad libitum.
License All animals used in this study were treated in accordance with the UK 1986 Animals (Scientific Procedures Act).
Procedure
Surgery Male Wistar rats were treated with desipramine (25 mg/kg ip, 30 minutes prior to 6-OHDA) to protect noradrenergic terminals. Rats were then anaesthetized in an induction chamber using isoflorane (1-2% in 95% O₂, 5% CO₂ carrier gas), placed in a Kopf stereotaxic frame and anaesthesia maintained with 0.5-1.0% isoflorane. An incision was made in the scalp and a 0.8-mm-diameter hole made in the skull at coordinates AP: −0.26 mm L: +2.0 mm mm (all coordinates taken from bregma). The neurotoxin 6-hydroxydopamine (6-OHDA) (8 μg free base in 4 μL of 0.9% saline containing 0.05% ascorbic acid) was injected into the left median forebrain bundle at a constant rate over 4 min (1 μl/min) using a 10-μL Hamilton syringe lowered to −8 mm below the dura. The needle remained in place for a further 4 min before being removed, and the wound cleaned and sutured. Flunixin hydrochloride (2.5 mg/kg, Dunlop's Veterinary Supplies, Dumfries, UK) was administered for pain relief and a rehydration treatment of 5% glucose in 0.9% saline (up to 5 ml ip) was given prior to recovery from the anaesthetic.

Behavioural Assessment

Confirmation of the Lesion
At least 2 weeks following surgery, animals were examined for rotational behaviour (see below) in response to the administration of apomorphine hydrochloride (0.5 mg/kg s.c. in 0.9% saline containing 0.05% ascorbic acid) to evaluate the extent of the lesion. Only those rats exhibiting >6 turns/min at peak activity were used in future studies.

Assessment of the Induction of Rotational Activity by Test Compounds
At least 1 week after apomorphine administration, rats (n=4-8 per treatment) were tested for rotational activity with either a test drug or L-DOPA. These were administered either via the intraperitoneal (ip) route or orally by gavage (po). Animals were treated with benserazide (10 mg/kg) and placed in rotometers (Med Associates) for up to 30 min to measure basal activity. They were then treated with test compound or L-DOPA (63.4 μmole/kg ip or po). Rotation behaviour was assessed for up to 4 hours after test drug/L-DOPA administration. Animals were typically treated with a series of compounds for comparative purposes. Each treatment was administered at least 1 day apart.

Data Analysis
The number of rotations measured per 10 minutes over the 4 hour period was determined. Animals were considered active if they turned >10 turns per 10 minutes. From this data the following parameters were measured:
   A Total activity (AUC activity, where AUC=area under the locomotor-activity/time curve)
   B Peak activity
   C Duration of activity
Values are quoted as % of L-DOPA induced effects.

Results:

| | | | Peak activity | duration of |
|---|---|---|---|---|
| | | AUC activity | (as % of | activity (as % of |
| | | (as % of AUC | maximal | duration of |
| example | route of | activity L- | activity L- | activity L- |
| number | admin. | DOPA) | DOPA) | DOPA) |
| 1 | p.o. | 40 | 53 | 45 |
| 2 | i.p. | 32 | 31 | 27 |
| 12 | p.o. | 50 | 62 | 46 |
| 13 | p.o. | 106 | 141 | 76 |

1. A compound of formula (I) or a salt, hydrate or solvate thereof: 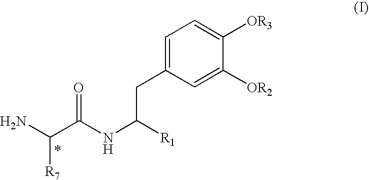
wherein:
R₁ is a carboxyl, carboxyl ester, or carboxamide group;
R₂ and R₃ are independently hydrogen, or a group —C(═O)R₆ or —C(═O)OR₆ wherein R₆ is C₁-C₆ alkyl, or a group —CH₂Q wherein Q is an optionally substituted monocyclic cycloalkyl or heterocyclyl ring of 3 to 6 ring atoms;
R₇ is (i) optionally substituted phenyl or monocyclic heteroaryl, or (ii) a radical of formula —CHR₄R₅;
R₄ is
(a) optionally substituted C₁-C₄ alkyl, C₁-C₄ alkoxy, C₂-C₄ alkenyl, C₂-C₄ alkenyloxy, or C₂-C₄ alkynyl, or
(b) —CH₂XCH₃, —CH₂CH₂XCH₃, or —CH₂XCH₂CH₃, wherein X is —O—, S, or —NR₇ wherein R₇ is hydrogen, methyl or ethyl; or —CH₂Q or CH₂OQ wherein Q is as defined in relation to R₆; and
R₅ is hydrogen, methyl, ethyl, or methyl substituted by 1, 2 or 3 fluoro atoms; or
R₄ and R₅ taken together with the carbon atom to which they are attached form an optionally substituted carbocyclic or heterocyclic ring of 3 to 6 ring atoms, optionally fused to a second, optionally substituted, carbocyclic or heterocyclic ring or 3 to 8 ring atoms;
PROVIDED THAT the group R₇ is not the side chain of a natural amino acid.

2. A compound as claimed in claim 1 wherein the stereochemical orientation of the bond marked * is S.

3. A compound as claimed in claim 1 wherein R₁ is a carboxyl group.

4. A compound as claimed in claim 1 wherein R₁ is a carboxyl ester group of formula —COOR^{C} wherein R^{C} is a C₁-C₆ alkyl or C₂-C₆ alkenyl group.

5. A compound as claimed in claim 4 wherein R^{C} is methyl or.

6. A compound as claimed in claim 1 wherein R₁ is —CONH₂.

7. A compound as claimed in claim 1 wherein R₂ and R₃ are each hydrogen.

8. A compound as claimed in claim 1 wherein R₂ and R₃ are independently —C(═O)R₆ or —C(═O)OR₆ wherein R₆ is methyl, ethyl, n- or isopropyl, tert-butylmethyl, or benzyl which is optionally substituted in the phenyl ring thereof.

9. A compound as claimed in claim 1 wherein R₇ is a radical of formula —CHR₄R₅ and R₄ is optionally substituted ethyl, n- or iso-propyl, n-, iso- or tert butyl, phenyl, naphthyl, benzyl, cyclopropyl, cyclobutyl, cyclopentyl, cyclohexyl, cyclopropylmethyl, cyclobutylmethyl, cyclopentylmethyl, cyclohexylmethyl, pyridyl, pyridylmethyl, piperidinyl, piperazinyl or morpholinyl.

10. A compound as claimed in claim 1 wherein R₇ is a radical of formula —CHR₄R₅ and R₅ is hydrogen.

11. A compound as claimed in claim 1 wherein R₇ is a radical of formula —CHR₄R₅ and R₄ and R₅ taken together with the carbon atom to which they are attached form an optionally substituted cyclopropyl, cyclobutyl, cyclopentyl, cyclohexyl, or piperidinyl ring.

12. A compound as claimed in claim 1 wherein R₇ is optionally substituted phenyl, pyridyl, thienyl, furyl, or pyrrolyl

13. A compound as claimed in claim 1 wherein any optional substituents are selected from methyl, trifluoromethyl, methoxy, trifluoromethoxy, cyclopropyl, halogen, cyano, hydroxy, mercapto, oxo, —NH₂, —NHR^{A}, or —NR^{A}R^{B} wherein R^{A} and R^{B} are independently methyl or ethyl.

14. A pharmaceutical composition comprising a compound as claimed in claim 1 together with a pharmaceutically acceptable carrier.

15. (canceled)

16. A method of treatment of a condition associated with impaired dopaminergic signalling in a subject, comprising administrating to the subject an amount of a compound as claimed in claim 1 effective to reduce such impairment of dopaminergic signalling.

17. The method as claimed in claim 16, wherein the condition is Parkinson's disease, or Restless Legs Syndrome

18. The method as claimed in claim 16, wherein the condition is Tourette's syndrome, attention deficit hyperactive disorder, generation of pituitary tumours, a parkinson-plus syndrome, levodopa responsive dystonia, dyskinesia, periodic movements in sleep, dysphagia or neuroleptic malignant syndrome.
